(19) United States
(12) Patent Application Publication
(10) Pub. No.: US 2007/0281007 A1
(43) Pub. Date: Dec. 6, 2007

(54) Mucoadhesive Oral Formulations of High Permeability, High Solubility Drugs

(76) Inventors: Jules S. Jacob, Taunton, MA (US); Peyman Moslemy, Providence, RI (US); Avinash Nangia, Sharon, MA (US); Ze'ev Shaked, San Antonio, TX (US); Mark Kreitz, Providence, RI (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 11/574,407

(22) PCT Filed: Aug. 29, 2005
(86) PCT No.: PCT/US05/30553

§ 371 Date: Feb. 27, 2007

Related U.S. Application Data
(60) Provisional application No. 60/604,990, filed on Aug. 27, 2004.
(60) Provisional application No. 60/607,905, filed on Sep. 8, 2004.
(60) Provisional application No. 60/650,191, filed on Feb. 4, 2005.

(51) Int. Cl. A61K 9/32; A61K 31/155; A61K 31/195; A61K 9/50; A61K 9/52; A61K 31/343; A61K 9/14

(52) U.S. Cl. 424/452; 424/462; 424/465; 424/469; 424/472; 424/473; 514/471; 514/561; 514/635

(57) ABSTRACT

Solid oral dosage formulations, such as tablet, mini-tab, multiparticulates or osmotic delivery systems, are coated with a mucoadhesive polymeric coating or formed of a mucoadhesive polymer to increase oral bioavailability of Biopharmaceutical Classification System (BCS) Class I drugs. Representative BCS I drugs include valacyclovir, gabapentin, furosemide, levodopa, metformin, and ranitidine HCl. The inclusion of mucoadhesives in the solid oral dosage form brings the dosage form into close proximity with the target epithelium and facilitates diffusion of drug into intestinal tissue. The mucoadhesive polymer may be either dispersed in the matrix of the tablet or applied as a direct compressed coating to the solid oral dosage form. Preferred mucoadhesive polymers include poly(adipic)anhydride “P(AA)” and poly(fumaric-co-sebacic)anhydride “P(FA:SA)”. Other preferred mucoadhesive polymers include non-erodable polymers such as DOPA-maleic anhydride co polymer; isopthalic anhydride polymer; DOPA-methacrylate polymers; and DOPA-cellulosic based polymers.

### CROSS-REFERENCE TO RELATED APPLICATIONS

This application claims priority to U.S. Ser. No. 60/604,990, entitled “Bioadhesive Rate Controlled Oral Dosage Formulation”, filed Aug. 27, 2004; U.S. Ser. No. 60/607,905, entitled “Mucoadhesive Oral Formulations of High Permeability, High Solubility Drugs”, filed Sep. 8, 2004 and U.S. Ser. No. 60/650,191, entitled “Mucoadhesive Oral Formulations of High Permeability, High Solubility Drugs”, filed Feb. 4, 2005.

### FIELD OF THE INVENTION

The present application is directed to the field of drug delivery, more specifically to improving bioavailability of BCS class I drugs.

### BACKGROUND OF THE INVENTION

The Biopharmaceutical Classification System (BCS), originally developed by G. Amidon, separates pharmaceuticals for oral administration into four classes depending on their solubility and their absorbability through the intestinal cell layer. According to the BCS, drug substances are classified as follows:

Class I—High Permeability, High Solubility

Class II—High Permeability, Low Solubility

Class III—Low Permeability, High Solubility

Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development. Class I drugs of the BCS system are highly soluble and highly permeable in the gastrointestinal (“GI”) tract.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. The permeability class boundary is based, directly, on measurements of the rate of mass transfer across human intestinal membrane, and, indirectly, on the extent of absorption of a drug substance in humans. The extent of absorption in humans reflects the fraction of dose absorbed, not the systemic bioavailability. It is measured using mass-balance pharmacokinetic studies, absolute bioavailability studies, intestinal permeability methods, in vivo intestinal perfusion studies in humans, and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). In the absence of evidence suggesting instability in the gastrointestinal tract, a drug is considered highly soluble when 90% or more of an administered dose, based on a mass determination or in comparison to an intravenous reference dose, is dissolved. A drug substance is considered highly permeable when the extent of absorption in humans is determined to be greater than 90% of an administered dose, based on mass-balance or in comparison to an intravenous reference dose. An IR drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

Examples of BCS class I drugs include those listed in Kasim et al. Mol. Pharmaceutics 1(1): 85-96 (2004) and Lindenberger et al. Eur. J. Pharm. Biopharm. 58(2):265-78 (2004), such as amitriptyline hydrochloride, biperiden hydrochloride, chloroquine phosphate, chlorpheniramine maleate, chlorpromazine hydrochloride, clomiphene citrate, cloxacillin sodium, cyclophosphamide, diazepam, doxycycline, ergotamine tartrate, fluconazole, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, salbutamol, warfarin sodium, caffeine, metoprolol, propranolol, theophylline, verapamil, valacylclovir, diltiazem, gabapentin, levodopa, and divalproex sodium.

Valacyclovir is an antiviral drug which is active against the Herpes viruses. It is used to treat infections with herpes zoster (shingles), herpes simplex genitalis (genital herpes), and herpes labialis (cold sores). Valacyclovir inhibits the replication of viral DNA which is necessary for viruses to reproduce themselves. Valacyclovir is converted to acyclovir in the body.

Gabapentin is a medication used for management of post-herpetic neuralgia (PHN). PHN is the pain that lasts one to three months after shingles has healed. Gabapentin is also used for the treatment of partial seizures in adults and children. Gabapentin is available in capsule, tablet, and oral solution forms. The mechanism of action of gabapentin is unknown, but it has been shown to display analgesic action and anticonvulsant activity. Despite being a Class I drug, gabapentin is not appreciably metabolized in humans. The bioavailability of gabapentin is not dosed proportionally; as the dose increases, the bioavailability of gabapentin decreases. At best, the bioavailability of gabapentin is 60% at a 900 mg dose, given three times a day. Food increases, only slightly, the rate and extent of absorption of gabapentin.

Many BCS Class I drugs, such as verapamil, levadopa, metformin, and gabapentin, are absorbed only in the upper small intestine and have little or no absorption in the distal small intestine or colon. Many BCS Class I drugs require specific transport carriers in the intestinal tissue for delivery. These carriers can be saturated, thereby preventing absorption of the drug and resulting in sub-optimal absorption.

A number of attempts have been made to increase oral bioavailability of drugs. For example, U.S. Pat. No. 4,167,558 to Sheth and Tossounian describes a floating dosage form with increased gastric residence time. Specifically, Sheth and Tossounian describe drugs encapsulated in hydrocolloids such as cellulose ethers, notably. hydroxypropylmethylcellulose. Encapsulated drug is released by diffusion into the gastric contents after swelling. Gerogianis et al., Drug. Dev. Ind. Pharm., 19:1061-1081 (1993) describes a floating dosage form in which the flotation properties are linked to increased molecular weight and viscosity of the polymers and reduced hydration of the polymers owing to chemical substitutions on the polymer side chains. Sangekar et al., Intl. J. Pharm., 35:187-91 (1987) compared a gastric floating dosage formulation to a non-floating formulation and demonstrated that gastric emptying of the dosage forms was related to food and not the floating properties of the dosage forms.

Although these formulations are theoretically effective, the rate of absorption is dependant on whether or not the patient ate when taking the drug. For example, the absorption of the drug is significantly higher when the drug is taken with a meal than when it is not. This may be due to competition between dissolution of drug, and aggregation of drug particles as the water-soluble material dissolves. The latter effect may be minimized in the presence of food.

U.S. Pat. No. 6,509,038 to Baert et al. notes these defects in the prior art formulations. Baert et al. advocate resolving these problems by melting the drug and a hydrophilic polymer together, at temperatures of up to 300° C., and then extruding the melted composition. However, ratios of 5 parts of polymer per one part of drug are needed, which makes it difficult to make tablets or capsules that can be swallowed by a patient.

Other known biologically-compatible hydrophobic polymers, such as polylactic-co-glycolic acid (p[LGA]) or polylactic acid (p[LA]), can be used to encapsulate micronized drugs. While these materials typically do not dissolve in water, they do form a coating that retards the rate of release from the matrix system. Such materials are often used to provide controlled-release formulations. However, a system containing a coating formed of a hydrophobic polymer may be especially sensitive to the rates of stomach and intestinal clearance, and thus affected by the timing of meals and other factors as well.

Therefore it is an object of the invention to provide drug formulations for oral administration with improved adsorption in the GI tract.

It is a further object of the invention to provide a method for making oral drug formulations with improved adsorption in the GI tract.

### SUMMARY OF THE INVENTION

Solid oral dosage formulations, such as tablet, mini-tab, multiparticulates or osmotic delivery systems, are coated with a mucoadhesive polymeric coating or formed of a mucoadhesive polymer to increase oral bioavailability of Biopharmaceutical Classification System (BCS) Class I drugs. The formulations may provide immediate release, controlled release, or a combination of immediate release with controlled release of the drug or drugs to be delivered. Representative BCS I drugs include valacyclovir, gabapentin, furosemide, levodopa, metformin, and ranitidine HCl. The inclusion of mucoadhesives in the solid oral dosage form brings the dosage form into close proximity with the target epithelium and facilitates diffusion of drug into intestinal tissue. The mucoadhesive polymer may be either dispersed in the matrix of the solid oral dosage form or applied as a direct compressed coating to the solid oral dosage form. Preferred mucoadhesive polymers include poly(adipic)anhydride “p[AA]” and poly (fumaric-co-sebacic) anhydride “p[FA:SA]”. Other preferred mucoadhesive polymers include non-erodable polymers such as DOPA-maleic anhydride co polymer; isopthalic anhydride polymer; DOPA-methacrylate polymers; and DOPA-cellulosic based polymers.

### BRIEF DESCRIPTION OF THE DRAWINGS

FIG. 1 is a cross-section of a trilayer tablet containing BCS I drugs in a central matrix. The inner core is surrounded on two sides by mucoadhesive polymer layers.

FIG. 2 is a cross-section of a longitudinally compressed tablet containing BCS Class I drugs and excipients, and optionally dissolution enhancers, composed in a single core matrix that is coated peripherally with a mucoadhesive polymer.

FIG. 3 is a longitudinal cross-section of a longitudinally compressed tablet containing BCS Class I drugs and excipients, and optionally dissolution enhancers, composed in a single monolithic layer (not shown) or multiple monolithic layers that is coated peripherally with a mucoadhesive polymer.

FIG. 4 is a cross-section of a longitudinally compressed tablet containing BCS Class I drugs and excipients, and optionally dissolution enhancers, composed in two (not shown) or three monolithic layers, which are separated by one or more plugs. The tablet is coated peripherally with a layer of mucoadhesive polymer. Optionally the tablet is first sealed entirely with a moisture-protective polymer and then coated peripherally with the mucoadhesive polymer.

FIG. 5 is a cross-section of a longitudinally compressed tablet that functions as an osmotic delivery system. The BCS Class I drugs and excipients, optionally including dissolution enhancers, are composed in a single core matrix.

FIG. 6 is a cross-section of a longitudinally compressed tablet that functions as push-pull, osmotic delivery system. The core contains one layer of drug and another layer of swelling polymer to push drug out of the tablet at controlled rates.

FIG. 7 is a cross-section of a longitudinally compressed tablet containing precompressed inserts of drug, excipients, and optionally permeation enhancers, embedded in a matrix of mucoadhesive polymer.

FIG. 8 is a cross-section of a longitudinally compressed tablet containing an active agent in the form of microparticles within the core.

FIG. 9 is a graph of time (hours) versus concentration of gabapentin in the plasma (μg/mL) for Gabapentin controlled release 400 mg (“Gabapentin XL”) (▪) and NEURONTIN® 400 mg (distributed by Parke Davis, a division of Pfizer, Inc.) (♦) tested in the fed dog model.

FIG. 10 is a graph of time (hours) versus concentration of valacyclovir in the plasma (μg/mL) for VALTREX® 500 mg (manufactured by GlaxoSmithKline) (♦), a controlled release formulation containing valacyclovir 500 mg (“Valacyclovir CR 500 mg”) (▪), and a controlled release formulation containing valacyclovir 400 mg plus 100 mg VALTREX® (manufactured by GlaxoSmithKline) (“Valacyclovir 400 mg CR+100 mg IR”) (▴) tested in the fed dog model.

FIG. 11 is a graph of time (hours) versus % of drug released from valacyclovir osmotic tablets (Lot #505-018) in 0.1 N HCl at 37±0.5° C., in the USP II dissolution apparatus at 100 rpm.

FIG. 12 is a graph is time (hours) versus % of drug released from valacyclovir tablets, providing release is a two-step release profile, (Lot #504-027) in 0.1 N HCl at 37±0.5° C., in the USP II dissolution apparatus at 100 rpm.

FIG. 13 is a graph of time (hours) versus % of drug released from sodium valproate tablets (Lot #507-063) in USP I apparatus using pH 6.8 PBS buffer at 100 rpm and 37° C.

FIG. 14 is graph of time (hours) versus % of drug released from sodium valproate tablets (Lot #507-064) in USP I apparatus using pH 6.8 PBS buffer at 100 rpm and 37° C.

FIG. 15 is a graph of time (hours) versus concentration of levodopa (∘) and carbidopa (●) in the plasma (ng/mL) for SINEMET® CR tablets (200 mg levodopa, 50 mg carbidopa) (manufactured by Bristol-Myers Squibb) tested in the fed dog model.

FIG. 16 is a graph of time (hours) versus concentration of levodopa (●) and carbidopa (∘) in the plasma (ng/mL) for bioadhesive trilayer tablets (200 mg levodopa, 50 mg carbidopa) tested in the fed dog model.

### DETAILED DESCRIPTION OF THE INVENTION

I. Compositions

The oral dosage formulations provide improved oral bioavailability for BCS Class I drugs. The formulations may provide immediate release, controlled release, or a combination of immediate release with controlled release of the drug or drugs to be delivered. Improved oral bioavailability of BCS Class I drugs is achieved by combining a drug with a mucoadhesive carrier. The mucoadhesive carrier may be applied as a coating or as part of the drug delivery matrix.

A. Drugs

Certain BCS class I drugs have good water solubility, and good intestinal permeability, but limited colonic absorption. Prolonged exposure to the lining of the GI tract enables drug delivery downstream to absorptive sites in the small intestine and colon. These drugs include amitriptyline hydrochloride, biperiden hydrochloride, chloroquine phosphate, chlorpheniramine maleate, chlorpromazine hydrochloride, clomiphene citrate, cloxacillin sodium, cyclophosphamide, diazepam, doxycycline, ergotamine tartrate, fluconazole, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, salbutamol, warfarin sodium, caffeine, metoprolol, propranolol, theophylline, verapamil, valacylclovir, diltiazem, gabapentin, levodopa, sodium valproate, and divalproex sodium. In the preferred embodiment, the drug is gabapentin.

B. Mucoadhesive Polymers

Mucoadhesive polymers are included in the formulation to improve gastrointestinal retention via adherence of the formulation to the walls of the GI tract. As used herein “mucoadhesive” generally refers to the ability of a material to adhere to a biological surface for an extended period of time. Mucoadhesion requires contact between a mucoadhesive material and a surface (e.g. tissue and/or cells). Thus the amount of mucoadhesive force is affected by both the nature of the mucoadhesive material, such as a polymer, and the nature of the surrounding medium. “Mucoadhesive polymers” are polymers that have an adherence to mucosal tissue of at least about 110 N/m² of contact area (11 mN/cm²). A suitable measurement method is set forth in U.S. Pat. No. 6,235,313 to Mathiowitz et al. Another suitable measurement method uses a Texture Analyzer TA XT II tensile tester, with pig intestine as the biological substrate. Polymer films on supports were prepared by dip-coating in concentrated polymers solution and drying. The films on supports can be air-dried for 24 hrs after dipping and lyophilized overnight to remove residual solvents. Pig intestine is cut into at least 1 in² sections, mounted into a perforated, plastic holder with the mucus side up and submerged in phosphate buffered saline (PBS, pH 6.8). A fresh piece of tissue is used for each test. A polymer-coated support is mounted on the Texture Analyzer, and brought into contact with the pig intestine sample. An uncoated support is typically used as the control. After a suitable period of time, such as 7 minutes, the support is lifted away from the sample tissue and the load versus deformation curve is plotted. Standard settings for the Texture Analyzer are: Pre-Test Speed of 0.50 mm/sec; Test Speed of 0.50 mm/sec; Post-Test speed of 0.50 mm/sec; applied force of 5.0 g; Time for applied force of 420.00 sec; Trigger force of 5.0 g; and Trigger distance of 0.000 mm.

In a preferred embodiment, mucoadhesive polymers are hydrophobic enough to be non-water-soluble, but contain a sufficient amount of exposed surface carboxyl groups to promote adhesion to mucosal tissue. These include, among others, non-water-soluble polyacrylates and polymethacrylates; polymers of hydroxy acids, such as polylactide, polyglycolide, and polycaprolactone; polyanhydrides; polyorthoesters; cellulosic polymers, such as ethylcellulose; blends of these polymers; and copolymers formed of the monomers of these polymers. Blending or copolymerization sufficient to provide a certain degree of hydrophilic character can be useful to improve wettability of the materials. For example, about 5% to about 20% of monomers may be hydrophilic monomers.

The mucoadhesive polymer may contain a hydrophobic polymeric backbone with mucophilic groups attached to the backbone. As used herein “mucophilic groups” means functional groups that create hydrogen bonds or ionic interactions with mucosal tissue. Examples of mucophilic groups include groups which contain carboxylic, hydroxyl, and/or catechol functionalities. The polymers that form the hydrophobic polymeric backbone typically have a solubility in water at neutral pH and standard temperature and pressure of less than 1 mg/mL. Suitable polymers for forming the hydrophobic polymeric backbone include polyacrylates and polymethacrylates, such as polymers available under the tradename EUDRAGIT® (distributed by Rohm America); polymers of hydroxy acids, such as polylactide, polyglycolide and polycaprolactone; polyanhydrides; polyorthoesters; polyalkenes, such as polyethylene and polypropylene; substituted polyalkenes, such as polystyrene; hydrophobic cellulosic polymers, such as ethylcellulose; blends of these polymers; and copolymers formed of the monomers of these polymers.

Preferably, the polymers are bioerodable, with preferred molecular weights ranging from 1000 to 15,000 kDa, and most preferably 2000 to 5000 Da.

Polyanhydrides are a preferred type of mucoadhesive polymer. Suitable polyanhydrides include poly (adipic anhydride) (“p[AA]”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

p[AA] is a surface-eroding polymer belonging to the polyanhydride family of bioerodable and biocompatible polymers. The polymer is thermoplastic polymer with a molecular weight ranging from 2 kDa to 50 kDa that quickly degrades to adipic acid and adipic anhydride (both of which are generally regarded as safe, i.e. “GRAS”, for food applications) over the course of 24 hours at physiological pH.

Other preferred mucoadhesive polymers include polylactic acid (2 kDa MW, types SE and HM), polystyrene, poly(bis carboxy phenoxy propane-co-sebacic anhydride) (20:80) (p[CCP:SA]), alginate (freshly prepared); and poly(fumaric anhydride-co-sebacic anhydride (20:80) (p[FA:SA]), types A (containing sudan red dye) and B (undyed). Other high-adhesion polymers include p[FA:SA] (50:50) and non-water-soluble polyacrylates and polyacrylamides.

Optionally, the polymer is a blend of hydrophilic polymers and hydrophobic polymers. Suitable hydrophilic polymers include hydrophilic cellulosic polymers, such as hydroxypropylmethylcellulose, hydroxypropylcellulose, and carboxymethylcellulose; polyvinylalcohols, polyvinylpyrollidones, and polyethylene glycols.

In a preferred embodiment, the mucoadhesive polymers contain a water insoluble hydrophobic backbone and mucophilic functional groups. A compound containing an aromatic group which contains one or more hydroxyl groups, such as catechol, can be grafted onto a polymer or coupled to individual monomers. The polymer or monomer that forms the polymeric backbone may contain accessible functional groups that easily react with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, acyl azides, isocyanates, isothiocyanates, and succinimidyl esters.

Mucoadhesive polymers may contain a catechol functionality. “Catechol” refers to a compounds containing a moiety with a molecular formula of C₆H₆O₂ and the following structure:

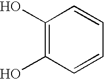

Monomers in the backbone of a polymer can be substituted with a compound containing a catechol functionality. The degree of substitution varies based on the desired adhesive strength. It may be as low as 10%, 20%, 25%, 50%, or up to 100% substitution. On average, at least 10% of the monomers in a suitable polymeric backbone are substituted with at least one aromatic group, preferably, on average, at least 10-20% of the monomers in the backbone are substituted with at least on aromatic group.

In the preferred embodiment, the aromatic compound containing one or more hydroxyl groups is catechol or a derivative thereof. Optionally the aromatic compound is a polyhydroxy aromatic compound, such as a trihydroxy aromatic compound (e.g. phloroglucinol) or a multihydroxy aromatic compound (e.g. tannin). The catechol derivative may contain a reactive group, such as an amino, thiol, or halide group. The preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g. by hydroxylation) to the DOPA form.

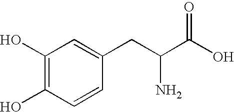

### 3,4-dihydroxyphenylalanine

In the preferred embodiment, the aromatic compound is an amine-containing aromatic compound, such as an amine-containing catechol derivative.

DOPA-containing mucoadehsive polymers include DOPA-maleic anhydride co-polymer, isopthalic anhydride polymer, DOPA-methacrylate polymers, DOPA-cellulosic based polymers, and DOPA-acrylic acid polymers.

Mucoadhesive materials available from Spherics, Inc., Lincoln, R.I., include SPHEROMER™ I (poly(fumaric acid:sebacic acid; p[FA:SA]), as described in U.S. Pat. No. 5,955,096 to Mathiowitz et al.), SPHEROMER™ II (anhydride oligomers, such as fumaric anhydride oligomer and metal oxides, such as CaO, ferric oxide, magnesium oxide, titanium dioxide, as described in U.S. Pat. No. 5,985,312 to Jacob et al.), and SPHEROMER™ III (L-DOPA grafted onto butadiene maleic anhydride at 95% substitution efficiency (L-DOPA-BMA), as described in WO 2005/056708 to Spherics, Inc.).

SPHEROMER™ II may be blended with methylmethacrylates, celluloses and substituted celluloses, polyvinylpyrollidones, PEGs, Poly (vinyl alcohols). Alternatively SPHEROMER™ II may be blended with other mucoadhesive polymers, such as p[FA:SA], p[AA], and L-DOPA-BMA.

In designing mucoadhesive polymeric formulations based on polylactides, polymers that have high concentrations of carboxylic acid are preferred. This can be accomplished by using low molecular weight polymers (Mw 2000 Da), since low molecular weight polymers contain high concentration of carboxylic acids at the end groups.

Excipients may also be added to improve mucoadhesion. Suitable excipients include FeO/Fe₂O₃, fumaric anhydride pre-polymer (FAPP), L-DOPA-L-DOPA dimer, and adipic anhydride pre-polymer (AAPP).

The BCS Class I drugs may optionally be encapsulated or molecularly dispersed in polymers to reduce particle size and increase dissolution. The polymers may include polyesters such as poly (lactic acid) (“p[LA]”), polycaprolactone, polylactide-co-glycolide (“p[LGA]”), poly hydroxybutyrate poly β-malic acid; polyanhydrides such as p[AA], p[FA:SA], poly (sebacic) anhydride (“p[SA]”); cellulosic polymers such as ethylcellulose, cellulose acetate, cellulose acetate phthalate; acrylate and methacrylate polymers such as EUDRAGIT® RS 100 (copolymer of acrylate and methacrylates with quaternary ammonium group, in the form of insoluble, pH independent granules, with low permeability), RL 100 (copolymer of acrylate and methacrylates with quaternary ammonium group, in the form of insoluble, pH independent granules, with high permeability), E100 and E PO (cationic polymer with a dimethylaminoethyl ammonium group, soluble in gastric fluid up to pH 5.0, sellable and permeable at pH above 5.0), L100-55 (anionic polymer of methacrylic acid and methacrylates with a —COOH group, in the form of a powder spray dried with L30 D-55), L100 (anionic polymer of methacrylic acid and methacrylates with a —COOH group, in the form of a powder that is soluble at pH above 6.0), S100 (anionic polymer of methacrylic acid and methacrylates with a —COOH group, in the form of a powder that is soluble at pH above 7.0) (distributed by Rohm America) or other polymers commonly used for encapsulation for pharmaceutical purposes and known to those skilled in the art. Also suitable are hydrophobic polymers, such as polyimides.

p[AA] prevents coalescence of drug domains within the spray-dried product resulting in increased drug surface area available for dissolution. Additionally, adipic acid monomer generated during polymer degradation increases acidity in the microenvironment of the spray-dried drug particle. By changing the pH, some of the drugs may become more soluble.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose.

C. Formulations

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The formulation may be in the form of a tablet, capsule, minitab, filled tablet, osmotic device, slurry, dispersion, or suspension. In the preferred embodiment, the formulation is a solid oral dosage formulation, such as a tablet, multiparticulate composition, or capsule.

The drug may be incorporated into a polymer matrix at any appropriate loading, such as from 1 to 90% w/w, from 1 to 50% w/w, 20 to 70% w/w, from 40 to 60% w/w, and preferably in a range from 20% to 30% w/w.

The active compounds (or pharmaceutically acceptable salts thereof) may be administered in a formulation wherein the active compound(s) is in an admixture with one or more pharmaceutically acceptable carriers, excipients or diluents. The pharmaceutical formulations may be produced using standard procedures.

The compounds may be complexed with other agents as part of the formulation. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (“Povidone”), HPMC, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If the resulting complex is water-soluble, then the complex may then be dissolved in an appropriate buffer, for example, phosphate buffered saline or other physiologically compatible solutions. Alternatively, if the resulting complex has poor solubility in aqueous solvents, then it may be formulated with a non-ionic surfactant such as a polyoxyethylene sorbitan fatty acid ester (e.g. TWEEN™), or polyethylene glycol. Thus, the compounds and their physiologically acceptable solvates may be formulated for administration.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

Coatings

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and acrylate and methacrylate polymers that are commercially available under the trade name EUDRAGIT® (distributed by Rohm America), zein, shellac, and polysaccharides.

Additionally, the coating material may contain conventional carriers such as plasticizers, pigments, colorants, glidants, stabilization agents, pore formers and surfactants.

Suitable coating materials are listed below in Table 1.

TABLE 1
Methacrylate-based coating materials

| Functionality | Trade name |
|---|---|
| Anionic polymer of methacrylic | EUDRAGIT ® L 100-55 - powder, spray dried L |
| acid and methacrylates with | 30 D-55 which can be reconstituted for targeted |
| a - COOH group | delivery in the duodenum |
| | EUDRAGIT ® L 30 D-55 - aqueous dispersion, pH |
| | dependent polymer soluble above pH 5.5 for |
| | targeted delivery in the duodenum |
| | EUDRAGIT ® L 100 - powder, pH dependent |
| | polymer soluble above pH 6.0 for targeted delivery |
| | in the jejunum |
| | EUDRAGIT ® S 100 - powder, pH dependent |
| | polymer soluble above pH 7.0 for targeted delivery |
| | in the ileum. |
| | EUDRAGIT ® FS 30 D - aqueous dispersion, pH |
| | dependent polymer soluble above pH 7.0, requires |
| | no plasticizer |
| Cationic polymer with a | EUDRAGIT E 100 - granules, pH dependent, |
| dimethylaminoethyl ammonium | soluble in gastric fluid up to 5.0, swellable and |
| group | permeable above pH 5.0. |
| | EUDRAGIT ® E PO - powder form of E-100 |
| Copolymers of acrylate | Insoluble, High Permeability |
| and methacrylates with | EUDRAGIT ® RL 30D - aqueous dispersion, pH |
| quarternary ammonium group | independent polymer for sustained release |
| | formulations |
| | EUDRAGIT ® RL PO - powder, pH independent |
| | polymer for matrix formulations |
| | EUDRAGIT ® RL 100 - granules, pH independent |
| | Insoluble, Low Permeability |
| | EUDRAGIT ® RS 30D - aqueous dispersion, pH |
| | independent polymer for sustained release |
| | formulations |
| | EUDRAGIT ® RS PO - powder, pH independent |
| | polymer for matrix formulations |
| | EUDRAGIT ® RS 100 - granules, pH independent |
| Copolymers of acrylate and | EUDRAGIT ® RD 100 - powder, pH independent |
| methacrylates with quarternary | for fast disintegrating films |
| ammonium group in combination | |
| with sodium | |
| carboxymethylcellulose | |

Suitable enteric coating materials include EUDRAGIT® 1-100-55, EUDRAGIT® L 100, EUDRAGIT® S 100, EUDRAGIT® FS 30 D, and ACRYL-EZE®, an aqueous enteric coating system distributed by Colorcon, which is soluble at pH 5.5.

Excipients

Optional pharmaceutically acceptable excipients present in the tablets, multiparticulate formulations, beads, granules, or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet, multiparticulate, bead, or granule remains intact during storage and until administration. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

Optionally, the tablets, beads, granules, or particles may also contain minor amounts of nontoxic auxiliary substances, such as wetting or emulsifying agents, dyes, pH buffering agents, or preservatives.

Preferred Oral Dosage Forms

In a preferred embodiment, the solid oral dosage form is a tablet, preferably a trilayer tablet, 10, containing BCS Class I drugs in a central matrix formed of pharmaceutically acceptable excipients, such as hydroxypropylmethylcellulose (“HPMC”) and microcrystalline cellulose (“MCC”) or spray-dried lactose, 12 (FIG. 1). The inner core is surrounded on two sides by a mucoadhesive polymer, such as DOPA-BMA polymer or a mixture of mucoadhesive p[FA:SA] polymer and EUDRAGIT® RS PO, 14. Optionally, the tablet is coated with an enteric coating, 16. The cross-section of this dosage form is illustrated in FIG. 1.

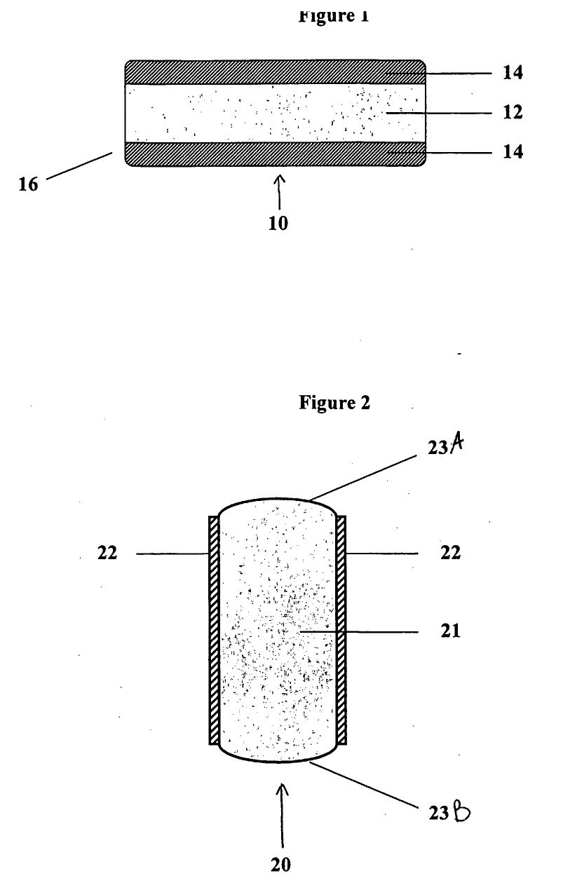

In another embodiment, the solid oral dosage form is a longitudinally compressed tablet, 20, containing BCS Class I drugs, excipients and, optionally, dissolution enhancers, composed in a single monolithic layer, 21. As used herein “monolithic layer” means that the layer is of uniform composition. The tablet is sealed peripherally with a layer of mucoadhesive polymer, 22, leaving the upper and lower sides, 23A and 23B, of the tablet available for drug release. First-order and, more advantageously, zero-order release profiles are achievable with this tablet design. It is feasible to create different release rates for drug by changing the composition of the core matrix. The cross-section of this dosage form is illustrated in FIG. 2.

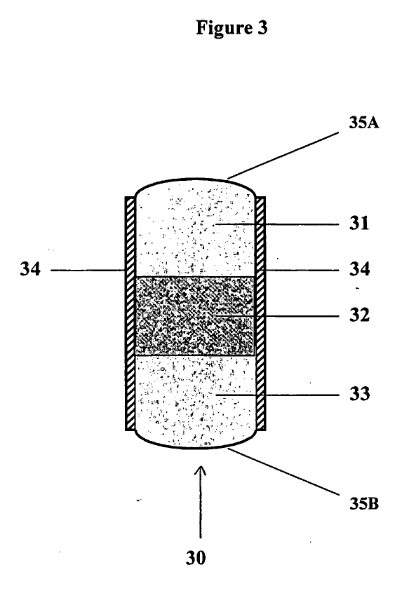

In another embodiment, the solid oral dosage form is a longitudinally compressed tablet, 30, containing BCS Class I drugs, excipients, and, optionally, dissolution enhancers, composed in a single monolithic layer or multiple monolithic layers, 31-33, which is sealed peripherally with a layer of mucoadhesive polymer, 34, leaving the upper and lower sides, 35A and 35B, of the tablet available for drug release. First-order and, more advantageously, zero-order release profiles are achievable with this tablet design. The tablet can be designed to provide immediate release of the drug and/or extended release rates for the drug by changing the composition of the core matrix or by changing the configuration of their respective layers. The cross-section of this dosage form is illustrated in FIG. 3.

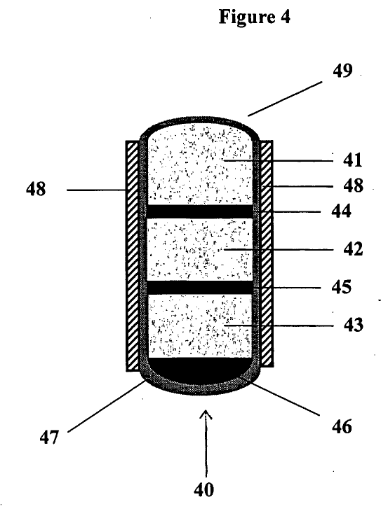

In another embodiment, the solid oral dosage form is a longitudinally compressed tablet, 40, containing BCS Class I drugs, excipients, and dissolution enhancers, composed in two or three monolithic layers, 41-43, which are separated by slow dissolving passive matrices (also referred to herein as “plugs”), 44-46. The tablet is coated entirely with a moisture-protective polymer, 47, and then sealed peripherally with a layer of mucoadhesive polymer, 48, leaving the upper side, 49, of the tablet available for drug release. First-order and, more advantageously, zero-order release profiles are achievable with this tablet design. The tablet can be designed to provide different immediate release or extended release rates for drugs in a two-pulse or three-pulse fashion by changing the composition or configuration of the drug layers, or by changing the formulation or configuration of the plugs. The cross-section of this dosage form is illustrated in FIG. 4.

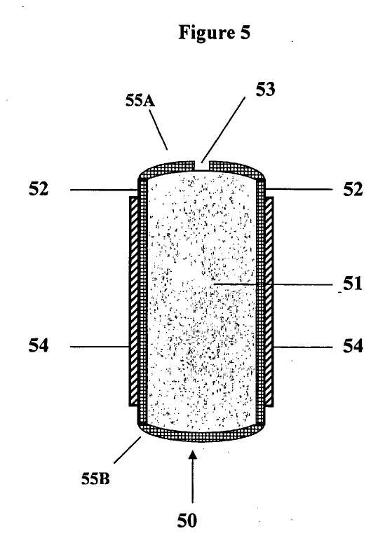

In another embodiment, the BCS Class I drug is delivered from an osmotic delivery system. FIG. 5 illustrates the cross-section of a longitudinally compressed tablet, 50, based on osmotic controlled delivery containing BCS Class I drugs, excipients, and, optionally, dissolution enhancers, composed in a single core matrix, 51. The tablet is coated with a semipermeable membrane, 52. One or both sides of the tablet may be perforated, such as by using a micro-drill or a laser beam to make a micrometer-sized orifice, 53. The tablet is sealed peripherally with a mucoadhesive polymer, 54, leaving the orifice and upper and/or lower sides, 55A and 55B, of the tablet available for drug release. The semipermeable membrane allows permeation of water into the matrix, leading to the dissolution of drug and creation of osmotic pressure. The increase of osmotic pressure will push the drug out of the device through the one or more orifice(s) and membrane at controlled rates. Zero-order release profiles are achievable with this tablet design.

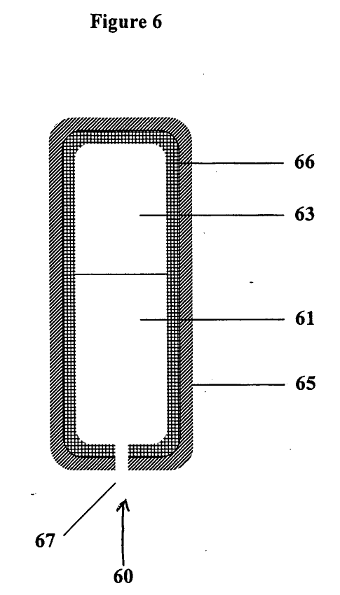

A cross section of an osmotic delivery system of the “push-pull” design is illustrated in FIG. 6. The osmotic delivery system is of the “push-pull” design, 60, and contains a micronized BCS Class I drug and osmotic agents, 61, to draw water across a semi-permeable membrane and a swelling polymer, 63, to push the drug out of the device at controlled rates. The entire device is coated with mucoadhesive polymers, 65, or contains polymer, 66, in the matrix of the capsule. As shown on FIG. 6, the mucoadhesive polymer can form a layer inside the capsule, 66; alternatively the mucoadhesive polymer can be included throughout the matrix containing drug in the capsule (not shown in FIG. 6). The tablet contains an orifice, 67, through which the drug is delivered.

In yet another embodiment illustrated in FIG. 7, a longitudinally compressed tablet, 70, containing precompressed inserts of drug and excipients, 74, and permeation enhancers and/or excipients, 72, is embedded in a matrix of mucoadhesive polymer. Drug is released only at the edge of the tablet and the kinetics of drug release is controlled by the geometry of the inserts. Zero and first-order release profiles are achievable with this tablet design and it is possible to have different release rates for permeation enhancer and drug by changing the configuration of their respective inserts.

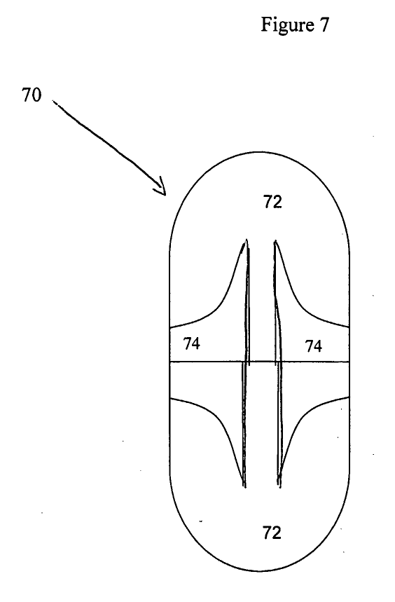

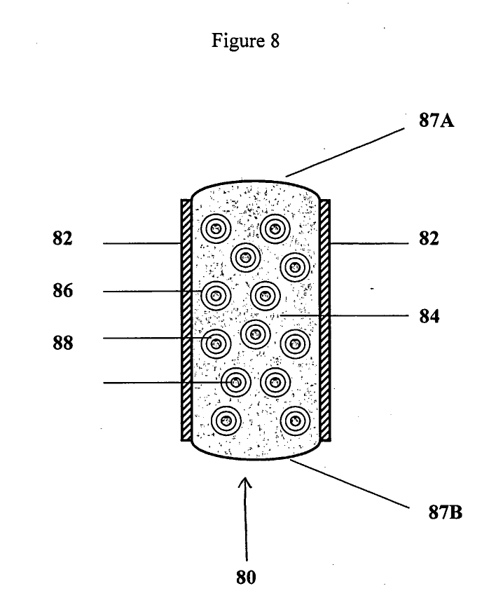

In yet another embodiment illustrated in FIG. 8, a longitudinally compressed tablet, 80, contains a mucoadhesive polymer layer, 82, and a core, 84, containing microparticles of active agent, 86. Optionally, and as illustrated in FIG. 8, the microparticles of active agent, 86, are coated with one ore more rate controlling polymers, 88. The tablet is sealed peripherally with a mucoadhesive polymer, 82, leaving the orifice and upper and/or lower sides, 87A and 87B, of the tablet available for drug release.

II. Methods of Making the Formulations

Solid oral dosage forms are typically prepared by blending powder drug or drug particles (i.e. drug in micro or nanoparticles) with excipients such as those discussed above and compressing the mixture into the form of a tablet. Alternately the mixture may be incorporated into standard pharmaceutical dosage forms such as gelatin capsules and tablets. Gelatin capsules, available in sizes 000, 00, 0, 1, 2, 3, 4, and 5, such as CAPSUGEL® (distributed by Capsugel®, a division of Pfizer), may be filled with mixtures and administered orally. Similarly, macrospheres may be dry blended or wet-granulated with diluents such as microcrystalline cellulose, lactose, cabosil and binders such as hydroxypropylmethylcellulose, hydroxypropylcellulose, carboxymethylcellulose and directly compressed to form tablets. The dimensions of the tablets are limited only by the engineering of dies available for tabletting machines. Dies to form tablets in round, oblong, convex, flat, and bullet designs in sizes ranging from 1 to 20 mm are available. The resulting tablets may weigh from 1 to 5,000 mg and carry macrospheres at loadings of 1 to 80% w/w.

The resulting tablets may be coated with sugars, enteric polymers or gelatin to alter dissolution of the tablet. Premature dissolution of the tablet in the mouth may be prevented by coating with hydrophilic polymers, such as hydroxypropylmethylcellulose or gelatin, resulting in dissolution in the stomach.

The tablet or solid oral dosage form may optionally contain absorption enhancers including: sodium caprate, ethylenediamine tetra (acetic acid) (EDTA), citric acid, lauroylcarnitine, palmitoylcarnitine, tartaric acid, Vitamin E tocopheryl polyethylene glycol succinate (TPGS), and other agents known to increase GI permeability by affecting integrity of tight junctions.

A. Formation of Drug Particles

The drug-polymer matrices may be fabricated using any of the encapsulation methods known to those skilled in the art, including but not limited to: solvent evaporation, solvent removal, spray-drying, phase-inversion encapsulation, spontaneous emulsification, coacervation, hot melt encapsulation, hot melt extrusion, spray-congealing, prilling and grinding. It is understood that the drug-polymer products may be further processed into oral dosage form using any of the standard pharmaceutical techniques including but not limited to tabletting, extrusion-spheronization and fluidized bed coating for multiparticulate dosage forms and capsule-filling.

The resulting particles are suitable for capsules, tableting and other conventional dosage forms.

1. Spray Drying

In one embodiment, the composition contains a drug/polymer mixture co-dissolved in a mutual solvent and then spray-dried to form microparticles in the range of 2-100 μm in diameter. Drug loadings can range from 0.5-60% (w/w) drug with polymer, but are typically in the range of about 30% to 40%. Polymer systems contain polymers with mucoadhesive qualities, and in the preferred embodiment may include either pure polyanhydride polymers, or mixtures of other biocompatible polymers (e.g., methacrylates, polyesters, polysaccharides) with polyanhydrides. The polymer system acts as a matrix for more rapid dissolution of the drug due to increased surface area by maintaining the micronized drug particle size. Spray dried polymer/drug product is then incorporated with suitable pharmaceutical excipients for capsule formation as an oral dose form.

2. Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent is evaporated, leaving solid particles. Several different polymer concentrations can be used, including concentrations ranging from 0.05 to 0.20 g/ml. The solution is loaded with a drug and suspended in 200 ml of vigorously stirred distilled water containing 1% (w/v) poly(vinyl alcohol) (Sigma). After 4 hours of stirring, the organic solvent evaporates from the polymer, and the resulting particles are washed with water and dried overnight in a lyophilizer. Particles with different sizes (1-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

3. Hot Melt Microencapsulation

In this method, the polymer is first melted and then mixed with the solid particles of dye or drug that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between one to 1000 microns are obtained with this method. The external surfaces of spheres prepared with this technique are usually smooth and dense. This procedure is used to prepare particles made of polyesters and polyanhydrides. However, this method is limited to polymers with molecular weights between 1000 and 50,000 Da.

4. Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

5. Extrusion-Spheronization

Core particles may be prepared by the process of granulation-extrusion-spheronization. In this process, micronized drug is mixed with microcrystalline cellulose, binders, diluents and water and extruded as a wet mass through a screen. The result is rods with diameters equal to the opening of the extrusion screen, typically in the size range of 0.1 to 5 mm. The rods are then cut into segments of approximately equal length with a rotating blade and transferred to a spheronizer. The spheronizer consists of a rapidly rotating, textured plate which propels rod segments against the stationary walls of the apparatus. Over the course of 1-10 minutes of spheronization, the rods are slowly transformed into spherical shapes by abrasion. The resulting spheroid cores are then discharged from the machine and dried at a temperature ranging from 40° C. to 50° C. for a time period ranging from 24 hours to 48 hours using tray-driers or fluidized bed dryers. The cores may then be coated with rate-releasing, enteric or mucoadhesive polymers using either pan-coating or fluidized-bed coating devices.

III. Methods of Making Bioadhesive Rate Controlling Oral Dosages

An extruded bioadhesive polymer cylinder is composed of one or more bioadhesive polymers, such as SPHEROMER™ I (poly(fumaric-co-sebacic) acid, as described in U.S. Pat. No. 5,955,096 to Mathiowitz et al.), SPHEROMER™ II (oligomers and metal oxides, as described in U.S. Pat. No. 5,985,312 to Jacob et al.), SPHEROMER™ III (DOPA side chains grafted onto a non-biodegradable polymeric backbone), poly adipic acid and 20% triethyl citrate (based on polymer weight) or other bioadhesive polymers available commercially along with suitable plasticizers, pore-forming agents, and solvents. Other thermoplastic polymers can be added to modify the moldability and mechanical strength of the bioadhesive polymer cylinder.

In the preferred embodiment, the extruded polymer cylinder is prepared using a hot-melt extrusion process, where the bioadhesive polymer is fed into the extruder as a pellet, flake, powder, etc., optionally along with a plasticizer. The materials are blended as they are propelled continuously along a screw through regions of high temperature and pressure to form the polymer extrudate. The extrudate is pushed from the extruded through a die having a suitable shape and dimension to form the desired cylinder. The cylinder is cooled after extrusion. The dimensions of the cylinder can be varied to accommodate the inner core system. The inner diameter of the cylinder can be configured to conform to the circumferential dimension of the preformed, pre-pressed inner system containing the therapeutic agent(s). The thickness of the cylinder is determined in part by the selected polymers and/or plasticizers as well its behavior with respect to the external fluid to which it is being delivered since the pH, volume, and flow conditions in different parts of the GI tract differ. For example, the small intestine has neutral pH, intermediate fluid volume and medium to high flow rates, while the large intestine has neutral pH, low fluid volume and low flow rates. The bioadhesive nature of the polymer cylinder may also be controlled by mixing different types of polymers and excipients. Inorganic metal oxides may be included in the cylinder to improve the adherence. Pore formers may also be added to control its porosity. Drugs may also be included into the polymer cylinder, optionally the drugs may act as a plasticizer or a pore former. Then the inner system, preferably in the form of longitudinally compressed tablet, is inserted into the cylinder, and the two components are fused together to resulting in a finished solid oral dosage form.

a. Production of the Hollow Bioadhesive Cylinder

Prior to hot-melt extrusion of the hollow cylinder, the bioadhesive polymer is mixed in a suitable device, such as a planetary mixer. Extrusion is performed using any suitable extruder, examples include a MP 19 TC25 laboratory scale co-rotating twin screw extruded of APV Baker (Newcastle-under-Lyme, UK) or a Killion extruder (Killian extruder Inc., Cedar Grove, N.J.). Both machines are equipped with a standard screw profile with two mixing sections, an annual die with metal insert for the production of the cylinder and twin screw powder feeder. Typical extrusion conditions are: a screw speed of 5 rpm, a powder feed rate of 0.14 kg/hr and a temperature profile of 125° C.-115° C.-105° C.-80° C.-65° C. from the powder feeder towards the die. The resulting hollow cylinders typically have the following dimensions: internal diameter of 7 mm, wall thickness of 1 mm and length of 1 cm.

b. Production of the Inner Core System

Inner longitudinally core tablets containing the therapeutic agent and other components are compressed onto a single or multilayer tableting machine equipped with deep fill or regular tooling. F or example, the therapeutic agent either alone or in combination with a rate controlling polymer and other excipients is mixed by stirring, ball milling, roll milling or calendaring and pressed into a solid having dimensions conforming to an internal compartment defined by the extruded polymer cylinder, so that it fits inside the cylinder. One or more layers containing different therapeutic agents can be included as a multilayer tablet. The inner core system may be a pre-fabricated osmotic system which is inserted into the bioadhesive cylinder with orifices aligned along the open ends of the cylinder.

c. Insertion of the Inner Core System into the Bioadhesive Cylinder

The preformed inner core with a diameter slightly smaller than the inner diameter of the cylinder is either manually or mechanically inserted into the cylinder and heated to fuse the two units. Alternately, the core may be inserted into the cylinder by a positive placement core insertion mechanism on a tableting machine. The extruded hollow cylinder may be placed into the die of a tableting machine. Then the compressed core may be placed inside the hollow cylinder. Next, the two components can be compressed to produce the finished solid dosage form. Alternatively, the dosage form may be prepared via simultaneous extrusion of the bioadhesive cylinder and an expandable inner composition using an extruder capable of such an operation.

IV. Uses of BCS Class I Formulations

The oral dosage formulations described herein can be used to treat a variety of diseases and disorders. Oral dosage forms described herein can administer antivirals to kill viruses. Additionally, anti-histimines, such chlorpheniramine maleate, can be administered to treat histamine response and other symptoms associated with allergies. Levodopa can be administered to treat Parkinson's disease. Anti-psychotic or antianxiety drugs, such as diazepam, and anti-epiliptic drugs, such as gabapentin and sodium valproate, can be administered to treat psychiatric disorders. Gabapentin can also be administered to treat depression. Antibiotics, such as doxycycline, can be administered to kill bacteria. Biperiden hydrochloride is an anti-cholinergic drug that can be administered to reduce tremors associated with Parkinson's disease. Fertility drugs, such as chlomiphene citrate, can be administered to treat infertility.

These formulations have improved bioavailability over formulations that do not contain the bioadhesive polymers. The formulations are designed to facilitate diffusion of drug into intestinal tissue.

The formulations can be designed to release drug slowly, quickly or in a step-wise (pulsatile) manner. For example, the dosage form can be designed to release drug linearly following administration, after a set time delay following administration, or in a multi-step manner, such as a two-step manner, or a three-step manner. Optionally, more than one drug is present in the formulation. Multi-drug formulations may be designed to release each drug simultaneously, or at different times. For example, the dosage form can administer the drugs in a two-step or three-step release, with a different drug released at each step.

The formulations may release at least 80% of the drug in 90 minutes, 4 hours, 12 hours, or up to 24 hours in vitro. The formulation may be designed to release at least 40% of the drug loaded in 30 minutes and at least 70% in 60 minutes in vitro.

The present invention will be further understood by reference to the following non-limiting examples.

### EXAMPLES

### Example 1

### Fluoroscopy Study of Barium-Impregnated Trilayer Tablets with Mucoadhesive Polymer Outer Layers

Trilayer tablets were prepared by sequentially filling a 0.3287×0.8937 “00 capsule” die (Natoli Engineering) with 333 mg of either SPHEROMER I™ or SPHEROMER III™ mucoadhesive polymer, followed by a layer of 233 mg of a blend of hydroxypropylmethylcellulose (HPMC) 4000 cps and 100 mg of barium sulfate, followed by an outer layer of 333 mg of either SPHEROMER I™ or SPHEROMER III™ mucoadhesive polymer. Trilayer tablets were prepared by direct compression at 2000 psi for 1 second using a Globepharma Manual Tablet Compaction Machine (MTCM-1).

The tablets were administered to female beagles that were fasted for 24 hrs (“fasted state”). The tablets were also dosed to fasted beagles that had been fed with chow, 30 minutes before dosing (“fed state”). Tablets were continuously imaged with fluoroscopy over the course of 6 hrs in unrestrained dogs. Typical results are indicated below. Trilayer tablets with SPHEROMER I™ or III™ in the mucoadhesive layers remained in the stomach of fasted dogs for up to 3.5 hrs and resided in the stomach of fed dogs in excess of 6 hrs. The tablets did not mix with food contents and remained in contact with stomach mucosa at the same location until they passed into the small intestine.

### Example 2

### Pharmacokinetics of Bioadhesive Gabapentin Tablets (“Gabapentin XL”) compared with NEURONTIN® (Gabapentin) (“Gabapentin IR”) in “Fed” Dog Model

Bioadhesive, trilayer tablets containing 400 mg gabapentin in the central core layer sandwitched between two bioadhesive layers, were compressed using 0.3287×0.8937″ capsule-shaped dies (Natoli Engineering) at 3000 psi for 3 seconds in a GlobePharma Manual Tablet Compaction Machine (MTCM-1). The composition of the inner core tablet and bioadhesive coating for the “Gabapentin XL” trilayer tablet are provided below in Tables 2 and 3.

TABLE 2
Composition of Active Core Layer in Gabapentin XL

| Component | Function | mg per tablet | % w/w |
|---|---|---|---|
| Gabapentin | Active Agent | 397 | 56.1 |
| hydroxypropyl | Rate-Controlling | 49 | 7.0 |
| methylcellulose 4000 cps | Polymer | | |
| hydroxypropyl | Rate-Controlling | 199 | 28.1 |
| methylcellulose 100 cps | Polymer | | |
| Microcrystalline cellulose | Filler/binder | 49 | 7.0 |
| (EMCOCEL ® 90M) | | | |
| Magnesium Stearate | Lubricant | 13 | 1.8 |

| | | |
|---|---|---|
| Total | 707 | 100.0 |

TABLE 3
Composition of Outer Bioadhesive Layers in Gabepentin XL

| Component | Function | mg per tablet | % w/w |
|---|---|---|---|
| Catechol-grafted | Bioadhesive Polymer | 450 | 90 |
| Butadiene Maleic | | | |
| Anhydride | | | |
| (SPHEROMER ™ III) | | | |
| polyvinylpyrrolidone | Binder | 45 | 9 |
| K-30 | | | |
| Magnesium Stearate | Lubricant | 5 | 1 |

| | | |
|---|---|---|
| Total | 500 | 100 |

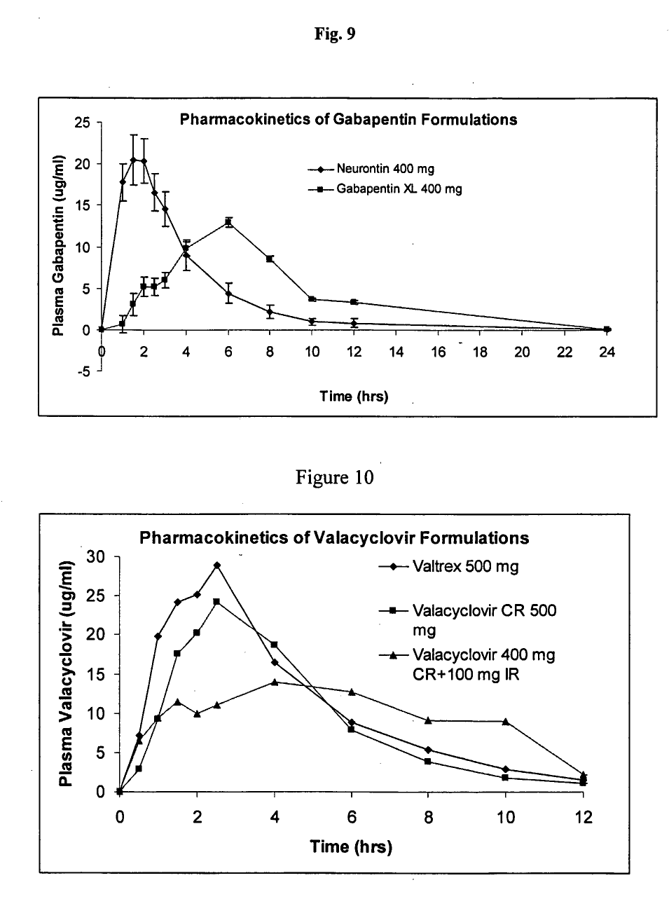

NEURONTIN® 400 mg, an immediate release gabapentin tablet (“Gabapentin IR”) distributed by Parke Davis, a division of Pfizer, Inc., was tested against the Gabapendin XL tablet. Gabapentin IR and Gabapentin XL tablets containing 400 mg of gabapentin were administered to cohorts of six beagle dogs in the fed state (as described in Example 1) and plasma levels of gabapentin were measured using LC/MS/MS (see FIG. 9).

The area under the plasma gabapentin vs. time curve (AUC), maximum concentration (Cₘₐₓ) and time required to achieve Cₘₐₓ (Tₘₐₓ) were calculated and the results are provided in Table 4 below.

TABLE 4
Pharmacokinetic Parameters for
Gabapentin IR and Gabapentin XL

| | AUC | Cₘₐₓ | Tₘₐₓ |
|---|---|---|---|
| Formulation | μg/ml * hr | μg/ml | hr |
| Gabapentin IR | 88.7 ± 14.0 | 22.9 ± 2.4 | 0.8 ± 0.3 |
| Gabapentin XL | 100.7 ± 11.2 | 16.3 ± 1.9 | 7.0 ± 1.2 |

Clearly, Gabapentin XL bioadhesive trilayer tablets were able to match and exceed the AUC of the immediate release form of gabapentin. Gabapentin is known be absorbed only in the upper small intestine and permeability is limited by carrier-mediated transport in intestinal mucosa. The higher Tmax of the bioadhesive, Gabapentin XL compared to the Gababentin IR, was characteristic of a controlled release formulation and indicative of gastroretention, based upon the narrow GIT absorption window of the drug.

### Example 3

### Comparison of Immediate Release Valacyclovir Tablets (VALTREX®) with Controlled Release Tablets in “Fed” Dog Model

Immediate Release Formulations

VALTREX® is the brand name for valacyclovir, a synthetic nucleoside analogue, manufactured by GlaxoSmithKline for treatment of diseases caused by Herpes virus. Valacyclovir is the prodrug for acyclovir and has greater solubility in water than acyclovir. The bioavailability of valacyclovir is ˜50% compared to ˜10-20% for acyclovir.

Controlled Release Formulations

Trilayer tablets described below (referred to as “CR 1” and “CR 2”) were identical in shape (0.3287×0.8937 “00 capsule”) and were compressed at 3000 psi for 5 seconds using the Globe Pharma MTCM machine.

Trilayer tablets were prepared according to the formulation listed below and were tested once (n=6/test) in the fed beagle model described in Example 1 and in simulated gastric fluid. The components of the inner core were blended but not granulated. Controlled Release formulation 1 (“CR 1”) was formulated as follows:

| | % w/w |
|---|---|
| Inner Core: (658 mg) | |
| Valacyclovir | 76.2 |
| ETHOCEL ® 10 Standard FP | 19.0 |
| (Ethyl cellulose, Dow Chemical Co.) | |
| Talc | 3.0 |
| AEROSIL ® | 0.6 |
| (hydrophilic fumed silica, Degussa AG) | |
| Magnesium Stearate | 1.1 |
| Outer Layer: (300 mg × 2) | |
| SPHEROMER ™ III | 99.0 |
| Magnesium Stearate | 1.0 |

Controlled Release formulation 2 (“CR 2”) was formulated using the same components in the same proportions as described above for CR 1, except that the inner core contained a total weight of 525 mg. A CR 2 tablet was placed in a hard gelatin capsule (CAPSULGEL®) along with 100 mg of Valacyclovir (VALTREX®, GlaxoSmithKline) to form a solid oral dosage form containing a total of 500 mg valacyclovir/dose (“CR 2 plus IR”).

Test in Fed Beagles

Female beagle dogs were fasted for 24 hrs and chow was returned 30 minutes before dosing (“fed state”) with 1 tablet of VALTREX® (Valacyclovir 500 mg), 1 tablet of CR 1, or 1 capsule containing CR2 plus IR.

FIG. 10 shows the pharmacokinetic profiles obtained for VALTREX®, CR 1, and CR 2 plus IR. Area under the plasma concentration versus time curve (AUC), maximum plasma concentration (Cmax) and time to maximum plasma concentration (Tmax) were calculated. The AUC, Cmax, and Tmax for each formulation (mean±standard error) is listed in Table 5.

TABLE 5
Pharmacokinetic parameters for VALTREX ® and
Controlled Release Formulations

| | AUC | Cmax | Tmax |
|---|---|---|---|
| Formulation | (μg/ml * hr) | (μg/ml) | (hr) |
| 500 mg VALTREX ® | 131.7 ± 13.8 | 33.8 ± 6.4 | 2.3 ± 0.5 |
| CR1 | 129.4 ± 15.7 | 26.8 ± 2.2 | 3.8 ± 1.0 |
| CR 2 plus IR | 133.7 ± 24.4 | 21.8 ± 13.9 | 4.3 ± 1.5 |

In Vitro Dissolution Data

VALTREX® 500 mg was tested for dissolution in simulated gastric fluid (SGF), pH 1.2 using the USP II apparatus at 100 rpm. 100% of valacyclovir in the VALTREX® tablets was released after 20 minutes in SGF.

CR 1 (500 mg) was tested for dissolution in simulated gastric fluid (SGF), pH 1.2 using the USP II apparatus at 100 rpm. 100% of valacyclovir in the CR 1 tablets was released after about 6 hours in SGF. CR 1 showed a nearly linear release of the acyclovir throughout the six hour time period.

### Example 4

### Longitudinally Compressed Osmotic Tablets Containing 500 mg Valacyclovir (Lot #505-018)

Longitudinally compressed tablets were prepared by using a special 0.2900″ die, two times longer than ordinary dies (Natoli Engineering). The die was filled with 700 mg of a dry blend of drug and excipients. A special punch, 2″ tip length, was used as the upper punch and to dislodge the tablets from the die. The tablets were prepared by direct compaction at 500 psi for 1 second using the GlobePharma Manual Tablet Compaction Machine (MTCM-1). Each tablet contained 500 mg Valacyclovir. The compositions of core tablets are provided below in Table 6.

The longitudinally compressed tablets were first coated completely with a cellulose acetate (CA 398-10) plus PEG 400 based semi-permeable coating. A passageway having a diameter of 500 μm was made on the cellulose acetate film on each side of the tablet using a micro-drill.

The tablets were coated peripherally with a single layer of semipermeable PCL film that was heat-sealed to the tablet core. Optionally, a bioadhesive polymer, such as SPHEROMER™ polymer layers comprising either SPHEROMER™ I (anhydride polymers), SPHEROMER™ II (anhydride oligomers blended with pharmaceutical polymers), SPHEROMER™ III (catechol-grafted anhydride polymers), or combinations of these polymers can also be applied over the semipermeable coating. The longitudinal cross-section of this dosage form, including the bioadhesive layer, 54, is illustrated in FIG. 6.

TABLE 6
Composition of Valacyclovir Core Tablet Formulation

| Ingredients | Weight (mg) |
|---|---|
| Valacyclovir HCl* | 555.8 |
| Hydroxypropyl cellulose | 141.4 |
| Magnesium Stearate | 2.8 |
| Total | 700.0 |

*Equivalent to 500 mg Valacyclovir

Coated tablets were tested for release profile in 0.1 N HCl at 37±0.5° C., in the USP II dissolution apparatus at 100 rpm. The in vitro dissolution profile of tablets is shown in FIG. 11.

### Example 5

### Longitudinally Compressed Pulsatile Delivery Tablets Containing 250 mg Valacyclovir (Lot #504-027)

Longitudinally compressed core tablets were prepared by using a pair of 0.2900″ dies (Natoli Engineering). The die was filled initially with 100 mg of ethylcellulose composition (Plug II), subsequently with 250 mg of a Valacyclovir immediate release (IR II) dry blend, followed with 100 mg of hydroxypropyl cellulose (Plug I), and finally with 150 mg of a Valacyclovir immediate release (IR I) dry blend. A 2″ tip length punch was used as the upper punch to dislodge the tablets from the die. The tablets were prepared by direct compaction at 500 psi for 1 second using the GlobePharma Manual Tablet Compaction Machine (MTCM-1). Each tablet contained 250 mg Valacyclovir HCl. The composition of core for each tablet is provided in Table 7.

The tablets were coated peripherally with a single layer of impermeable PCL film that was heat-sealed to the tablet core. Optionally, one or more bioadhesive polymers, such as Spheromer™ polymer comprising either Spheromer I (anhydride polymers), Spheromer II (anhydride oligomers blended with pharmaceutical polymers), Spheromer III (catechol-grafted anhydride polymers), or combinations of these polymers can be applied over the impermeable coating.

TABLE 7
Composition of Valacyclovir, 250 mg Core Tablet Formulation

| | Weight (mg) | | | |
|---|---|---|---|---|
| Ingredients | IR I | Plug I | IR II | Plug II |
| Valacyclovir HCl* | 138.9 | — | 138.9 | — |
| Compressible Sugar | 103.85 | — | 103.85 | — |
| Croscarmellose Sodium | 6.25 | — | 6.25 | — |
| Magnesium Stearate | 1.00 | — | 1.00 | — |
| Hydroxypropyl Cellulose | — | 100.0 | — | — |
| Ethylcellulose | — | — | — | 100.0 |
| Total | 250.0 | 100.0 | 250.0 | 100.0 |

*Equivalent to 250 mg Valacyclovir

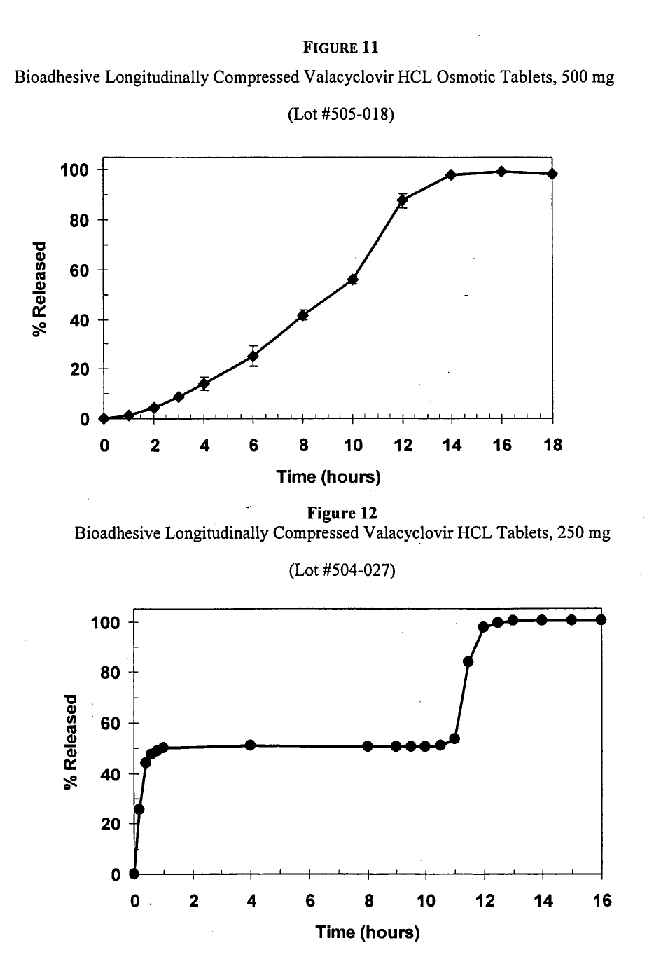

Coated tablets were tested for release profile in 0.1 N HCl at 37±0.5° C., in the USP II dissolution apparatus at 100 rpm. The in vitro dissolution profile of tablets is shown in FIG. 12. As shown in FIG. 12, this dosage form results is a two-step release of the drug.

### Example 6

### Sodium Valproate Bioadhesive Trilayer Tablets Based on Concentration Gradient Approach

Two different lots of sodium valproate bioadhesive tablet formulations (Lot #507-063 and 507-064), based on the concentration gradient approach, were prepared. Tablets from lot #507-063 utilized the L-DOPA/BMA polymer (SPHEROMER™ III) as the bioadhesive polymer, and tablets from lot #507-064 contained (FA:SA) bioadhesive polymer. An additional trilayer tablet (lot #507-065) using ethyl cellulose as a non-bioadhesive polymer was also prepared. Following steps were used to make the three lots.

Granulation of Sodium Valproate

180.0 g of sodium valproate (Katwijk Chemie BV) was granulated using a binder solution prepared previously by dissolving 10 g of ethyl cellulose (10-FP, NF Premium) and 10 g of polyvinylpyrrolidone, K-15 in 667 mL of ethanol. Binder solution was applied onto the drug in the bench top fluidized-bed spray-coating unit (Vector Corp. model MFL.01).

The following process parameters were used: fluid bed N₂ gas-flow=60-140 LPM; spray-nozzle pressure=15 psi; inlet temperature=50° C.; exhaust temperature=21-26° C.; pump speed=40 rpm; screen size=“I”; Wurster partition=medium; spray=bottom spray; and spray nozzle=medium. The granulation was dried and blended with 1% colloidal silicon dioxide. The granulation was stored in a 1-Liter glass jar containing DesiPak dessicant until used.

Blend: The sodium valproate granulation was blended with various excipients to form the sodium valproate inner and outer layers compositions as described below in Tables 8 and 9. The granulation was initially blended with ethyl cellulose, SPHEROMER™ I (p (FA:SA)) or SPHEROMER™ III (DOPA grafted on BMA) in a blender for 5 minutes followed by blending with magnesium stearate for additional 5 minutes.

TABLE 8
Composition of Common Inner Layer Blend

| Ingredients | Weight (%) |
|---|---|
| Sodium Valproate Granulation | 59.0 |
| Ethyl Cellulose, 10FP | 40.0 |
| Magnesium Stearate | 1.0 |
| Total | 100.0 |

TABLE 9
Composition of Outer Layer Blends

| Ingredients | Weight (%) |
|---|---|
| Sodium Valproate Granulation | 7.65 |
| SPHEROMER ™ I or | 91.35 |
| SPHEROMER ™ III | |
| Magnesium Stearate | 1.0 |
| Total | 100.0 |

Trilayer Tablets: Trilayer tablets were compressed on a Globepharma MTCM-1 manual tablet press using the 0.328″×0.8937″ capsule shaped, deep concave punches. First, 200 mg of outer blend was added to the die cavity and pre-compressed, then 987.2 mg of inner blend was added to the die cavity and pre-compressed again, and finally the 200 mg outer blend was added and compressed at 3000 psi for 1 s.

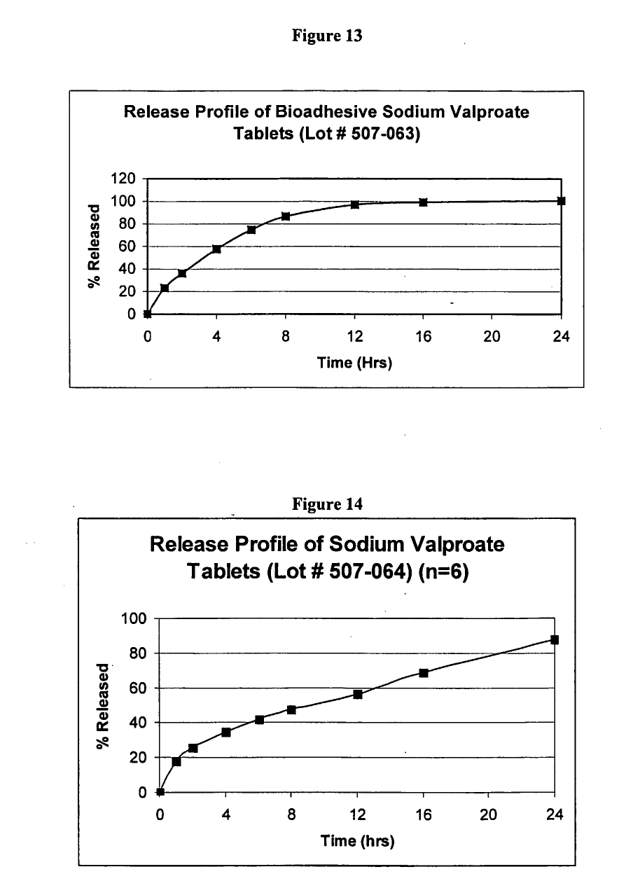

In vitro Dissolution Testing: Trilayer tablets were tested for dissolution testing in USP I apparatus using pH 6.8 PBS buffer at 100 rpm and 37° C. The dissolution profiles for three lots are shown in FIGS. 13 and 14.

### Example 7

### Comparison of Release of Drug from SINEMET® CR Tablets (Manufactured by Bristol-Myers Squibb) and Bioadhesive Trilayer Tablets, Each Containing 200 mg Levodopa and 50 mg Carbidopa

SINEMET® CR Tablets (manufactured by Bristol-Myers Squibb) containing 200 mg of levodopa and 50 mg of carbidopa (Lot # N4682) were placed in 0.1N HCl at 37±0.5° C., in the USP II dissolution apparatus at 50 rpm. The tablets had released all of the levodopa and carbidopa after about 3 hours following placement in the dissolution apparatus.

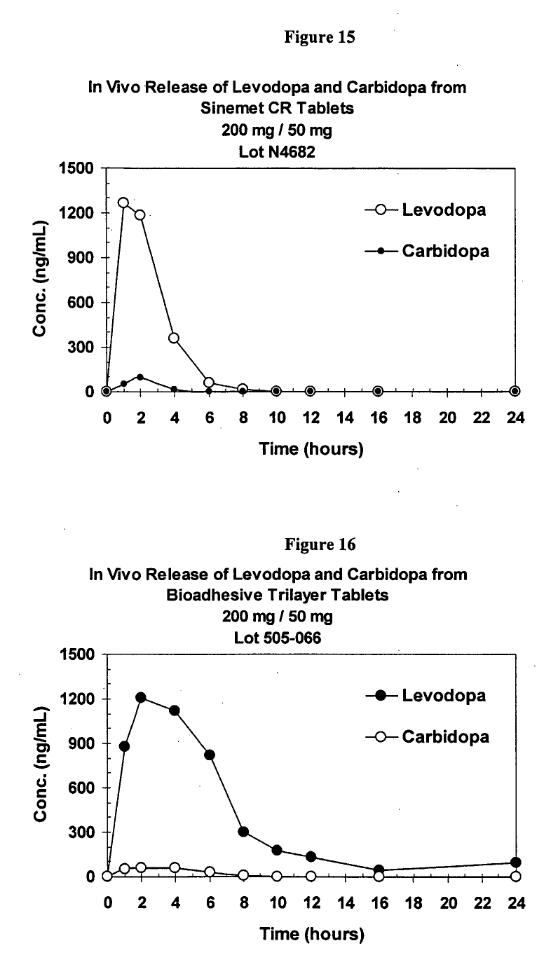

The tablets were orally administered to beagle dogs that had been fed with PROPLAN® Dry Dog Food-Adult (Purina) 30 minutes before dosing. The variation of concentration of both levodopa and carbidopa in the dogs' plasma is depicted in FIG. 15. The values of Tmax, Cmax, and AUC (area under the concentration vs. time curve) for the levodopa in the dogs' plasma were 1 hr, 1262.3 ng/mL, and 3903.0 ng*hr/mL, respectively.

Trilayer tablets were prepared by sequentially filling a 0.3287″×0.8937 ″ “00 capsule” die (Natoli Engineering) with 250 mg of Spheromer™ III bioadhesive polymer composition, followed by a layer of 466.7 mg of a blend of levodopa, carbidopa and pharmaceutically acceptable excipients, followed by an outer layer of 250 mg of Spheromer™ III bioadhesive polymer composition. Trilayer tablets were prepared by direct compression at 3000 psi for 1 second using a GlobePharma Manual Tablet Compaction Machine (MTCM-1).

Each tablet contained 200 mg of levodopa and 54 mg of carbidopa monohydrate, which is equivalent to 50 mg Carbidopa anhydrous. The core composition of tablet is provided in the Table 10.

TABLE 10
Composition of Bioadhesive Trilayer Tablets

| | Weight (mg) | | |
|---|---|---|---|
| | Outer | | |
| Ingredients | Layer 1 | Core Layer | Outer Layer 2 |
| Levodopa | — | 200.0 | — |
| Carbidopa, Monohydrate* | — | 54.0 | — |
| Hydroxypropyl | — | 167.2 | — |
| methylcellulose 100 cps | | | |
| Hydroxypropyl | — | 20.9 | — |
| methylcellulose E5 Prem LV | | | |
| L-Glutamic Acid HCl | — | 10.4 | — |
| Corn Starch | — | 10.4 | — |
| SPHEROMER ™ III | 245.0 | — | 245.0 |
| Ethyl cellulose 100 Std FP | 2.5 | — | 2.5 |
| Magnesium Stearate | 2.5 | 3.8 | 2.5 |
| Total | 250.0 | 466.7 | 250.0 |

*Equivalent to 50 mg Carbidopa Anhydrous

Bioadhesive trilayer tablets were placed in 0.1N HCl at 37±0.5° C., in the USP II dissolution apparatus at 50 rpm. The tablets had released all of the levodopa and carbidopa after about 16 hours following placement in the dissolution apparatus.

The trilayer tablets were orally administered to beagle dogs that had been fed with PROPLAN® Dry Dog Food-Adult (Purina) 30 minutes before dosing. The variation of concentration of both Levodopa and Carbidopa in the dogs' plasma is depicted in FIG. 16. The values of Tmax, Cmax, and AUC (area under the concentration vs. time curve) for the levodopa in the dogs' plasma were determined to be 2 hr, 1210.8 ng/mL, and 8536.7 ng*hr/mL, respectively.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

1. An oral formulation comprising a Class I drug in a matrix and a mucoadhesive polymer, wherein the mucoadhesive polymer forms a coating on at least part of the outside surface of the oral formulation.

2. The formulation of claim 1, wherein the mucoadhesive polymer comprises a water insoluble hydrophobic backbone and mucophilic functional groups.

3. The formulation of claim 2 wherein the hydrophobic polymer is selected from the group consisting of polyanhydrides, poly(meth)acrylates, polyhydroxy acids, polyesters, and copolymers thereof.

4. The formulation of claim 3 wherein the polymer is a polyanhydride.

5. The formulation of claim 2, wherein the mucophilic groups are selected from the group consisting of carboxylic, hydroxyl, and catechol functionalities, and combinations thereof.

6. The formulation of claim 5, wherein the catechol group is 3,4 dihydroxyphenylalanine (DOPA).

7. The formulation of claim 1, wherein the drug is selected from the group consisting of gabapentin, valacyclovir, furosemide, levodopa, metformin, and ranitidine hydrochloride.

8. The formulation of claim 7, wherein the drug is gabapentin.

9. The formulation of claim 1, wherein the formulation is a solid oral dosage formulation selected from the group consisting of tablets, capsules, minitabs, filled tablets, and osmotic tablets.

10. The formulation of claim 1, wherein the drug is in the form of particles.

11. The formulation of claim 1, further comprising a permeation or absorption enhancer.

12. The formulation of 11, wherein the enhancer is selected from the group consisting of sodium caprate, ethylenediamine tetra(acetic acid) (EDTA), citric acid, lauroylcarnitine, palmitoylcarnitine, tartaric acid, Vitamin E, and tocopheryl polyethylene glycol succinate.

13. The formulation of claim 9, wherein the solid oral dosage formulation is in a form selected from the group consisting of trilayer tablets and longitudinally compressed tablets.

14. The formulation of claim 9, wherein the solid oral dosage formulation is an immediate release formulation, a controlled release formulation or a combination thereof.

15. (canceled)

16. (canceled)

17. The formulation of claim 13, wherein the longitudinally compressed tablet is composed of a single monolithic layer or multiple monolithic layers.

18. (canceled)

19. The formulation of claim 17, wherein the longitudinally compressed tablet is composed of multiple monolithic layers and comprises at least one controlled release layer and at least one immediate release layer.

20. The formulation of claim 1 wherein the mucoadhesive coating is surrounded with an enteric-coating or non-enteric coating polymer.

21. A method of enhancing oral bioavailability of a BSC class 1 drug comprising providing the drug in an oral formulation comprising a Class I drug in a matrix and a mucoadhesive polymer, wherein the mucoadhesive polymer forms a coating on at least part of the outside surface of the oral formulation.

22. A method for treating a patient in need thereof comprising, administering to the patient an oral formulation comprising a Class I drug in a matrix and a mucoadhesive polymer, wherein the mucoadhesive polymer forms a coating on at least part of the outside surface of the oral formulation.

23. The method of claim 21, wherein the drug is selected from the group consisting of gabapentin, valacyclovir, furosemide, levodopa, metformin, and ranitidine hydrochloride.
